(19) United States
(12) Patent Application Publication
(10) Pub. No.: US 2007/0254088 A1
(43) Pub. Date: Nov. 1, 2007

(54) COMPOSITION COMPRISING ONE OR MORE ESTERIFIED PHYTOSTEROLS AND/OR PHYTOSTANOLS INTO WHICH ARE SOLUBILIZED ONE OR MORE UNESTERIFIED PHYTOSTEROLS AND/OR PHYTOSTANOLS, IN ORDER TO ACHIEVE THERAPEUTIC AND FORMULATION BENEFITS

(75) Inventors: David John Stewart, North Vancouver (CA); Jerzy Zawistowski, Port Moody (CA); Miruna Laza, Vancouver (CA)

Correspondence Address: KIRTON AND MCCONKIE, 60 EAST SOUTH TEMPLE,, SUITE 1800, SALT LAKE CITY, UT, 84111, US

(73) Assignee: FORBES MEDI-TECH INC., Vancouver, BC (CA)

(21) Appl. No.: 11/743,089

(22) Filed: May 1, 2007

Related U.S. Application Data
(60) Provisional application No. 60/796,985, filed on May 1, 2006.

(51) Int. Cl. A23D 9/00

(52) U.S. Cl. 426/611

(57) ABSTRACT

A composition comprises one or more free (unesterified) phytosterols and/or phytostanols which are substantially completely dissolved in one or more esterified phytosterols and/or phytostanols. The present invention further provides a composition comprising one or more esterified phytosterols and/or phytostanols which are substantially completely dissolved in an edible oil.

### FIELD OF THE INVENTION

This present invention relates to the field of supplementing foods and nutraceuticals with phytosterols and phytostanols and their esters.

### BACKGROUND OF THE INVENTION

While recent advances in science and technology are helping to improve quality and add years to human life, the prevention of atherosclerosis, the underlying cause of cardiovascular disease (“CVD”) has not been sufficiently addressed. Atherosclerosis is a degenerative process resulting from an interplay of inherited (genetic) factors and environmental factors such as diet and lifestyle. Research to date suggest that cholesterol may play a role in atherosclerosis by forming atherosclerotic plaques in blood vessels, ultimately cutting off blood supply to the heart muscle or alternatively to the brain or limbs, depending on the location of the plaque in the arterial tree^{1,2}. Data from the early Framingham Epidemiological Study indicates that increases in serum cholesterol levels are associated with increased risk of death from CVD³. More recent studies confirm that CVD is a leading cause of death and disability in industrialized nations⁴.

Studies have indicated that a 1% reduction in a person's total serum cholesterol yields a 2% reduction in risk of a coronary artery event⁵. Statistically, a 10% decrease in average serum cholesterol (e.g. from 6.0 mmol/L to 5.3 mmol/L) may result in the prevention of 100,000 deaths in the United States annually⁶.

As the population becomes increasingly aware of the importance of maintaining cholesterol balance in check, the need for naturally derived, safe and effective agents which address the underlying causes of CVD, and which can be readily incorporated into a wide variety of delivery means, becomes even more apparent.

One focus of such research related to naturally derived, safe and effective agents to address the underlying causes of CVD has been plant-derived sterols and stanols (also known as phytosterols and phytostanols). Sterols are naturally occurring compounds that perform many critical cellular functions. Phytosterols such as campesterol, stigmasterol and beta-sitosterol in plants, ergosterol in fungi and cholesterol in animals are each primary components of cellular and sub-cellular membranes in their respective cell types. The dietary source of phytosterols in humans comes from plant materials i.e. vegetables and plant oils. The estimated daily phytosterol content in the conventional western-type diet is approximately 60-80 milligrams in contrast to a vegetarian diet which would provide about 500 milligrams per day.

Phytosterols have received a great deal of attention due to their ability to decrease serum cholesterol levels when fed to a number of mammalian species, including humans. While the precise mechanism of action remains largely unknown, the relationship between cholesterol and phytosterols is apparently due in part to the similarities between the respective chemical structures (the differences occurring in the side chains of the molecules). It is assumed that phytosterols displace cholesterol from the micellar phase and thereby reduce its absorption or possibly compete with receptor and/or carrier sites in the cholesterol absorption process.

Over forty years ago, Eli Lilly marketed a sterol preparation from tall oil and later from soybean oil called Cytellin™ which was found to lower serum cholesterol by about 9% according to one report.⁷ Various subsequent researchers have explored the effects of sitosterol preparations on plasma lipid and lipoprotein concentrations⁸ and the effects of sitosterol and campesterol from soybean and tall oil sources on serum cholesterols.⁹ Compositions have been explored in which phytosterols or phytostanols (their hydrogenated counterparts) are esterified in order to enhance solubility. One composition of phytosterols which has been found to be highly effective in lowering serum cholesterol is disclosed in U.S. Pat. No. 5,770,749 to Kutney et al.

Despite the obvious and now well recorded advantages of phytosterols, not only in the treatment of CVD and its underlying conditions such as hypercholesterolemia, hyperlipidemia, atherosclerosis, hypertension, thrombosis but in the treatment of other diseases such as Type II diabetes, dementia cancer and aging, the administration of phytosterols and the incorporation thereof into foods, pharmaceuticals and other delivery vehicles has been complicated by the fact that they are highly hydrophobic (i.e. they have poor water solubility). This highly hydrophobic nature of phytosterols renders them insoluble and barely dispersible in aqueous media. As such, phytosterols tend to be added to the fat phase of fat-based food products. Health-conscious consumers wishing to benefit from the cholesterol lowering effects of phytosterols are therefore forced to consume fat-rich foods, despite the health risks of a high fat diet.

In addition, and critically in the area of food and beverage production, free, unesterified phytosterols have a waxy consistency and a high melting point, creating solubility issues for the food processor. While they are oil-dispersible to some extent in their raw form, the amount required to produce an efficacious effect in a finished product can cause granulation. The current answer to this problem is esterification, which creates something of an equilibrium between the phytosterols and liquid oil. Due to these physical property limitations of phytosterols, their use by food manufacturers has been limited to fat-based products like margarine, salad dressings and, most recently, snack bars.

Furthermore, studies have investigated how the form (for example crystalline, suspension, granular) in which the phytosterols are dosed impacts on their ability to lower serum cholesterol levels. As phytosterols are highly hydrophobic, they do not dissolve to any appreciable extent in the micellar phase in the digestive tract and therefore are not capable of efficiently blocking cholesterol absorption. Oils and fats are capable to a limited but not satisfactory degree of dissolving free phytosterols. Only substantially solubilized phytosterols appear inhibit the absorption of cholesterol.

As noted above, since phytosterols have high melting points (typically about 136-150° C.) it is important to maintain a temperature of 80° C. or higher during dissolution of phytosterols in fats or oils, in order to avoid recrystallization of the phytosterols. Crystalline phytosterol imparts an unpleasant grainy, waxy texture to edible and topical products. However, at 80° C. and above commonly used fats and oils are vulnerable to oxidation. Rancid oils and fats detract greatly from the organoleptic properties of food products in particular. Hence there is a need to address the issue of this waxy texture in order to make any deliverable foods and beverages palatable and marketable.

Early research on phytosterols focused on grinding or milling the phytosterols in order to enhance their solubility (U.S. Pat. Nos. 3,881,005 and 4,195,084 both to Eli Lilly). In addition, researchers have looked to the esterification of phytosterols in order to enhance their solubility. German Patent 2035069/Jan. 28, 1971 (analogous to U.S. Pat. No. 3,751,569) describes the addition of phytosterol fatty acid esters to cooking oil. The esterification is carried out between a free sterol and a fatty acid anhydride, with perchloric acid as the catalyst. The significant drawback to this process, along with others, is the use of non-food grade catalysts and reagents.

Conventionally, phytosterols have been incorporated into food products by melting a sterol or stanol, incorporating it into an oil phase, and blending the oil phase with other components to result in a phytosterol-containing food product. However, the aforementioned high melting points can result in significant crystallization of the phytosterols within the oil phase of such food products. Such crystallization results in food products with a gritty and unacceptable texture. This gritty texture is especially detectable when the oil/plant sterol phase is incorporated at high levels in the food product. The high melting points and hydrophobic nature of such phytosterols also make it difficult to blend such them with an aqueous phase. Furthermore, actual melting of the plant sterol for incorporation into food products is energy intensive.

Attempts have been made to solve these problems using, for example, chemical modification of the phytosterols. For example, as noted above, esterification of phytosterols generally results in lowered melting temperatures. Thus, such phytosterol esters generally may be incorporated into food products more readily due to the lower melting points and can provide food products without significantly gritty texture. Although the problem of fat solubility of phytosterols can be improved by esterification, this is not a completely satisfactory solution to the problem for various reasons, as described further below.

Although phytosterols and phytostanols, and their respective free or ester forms have similar efficacy in lowering LDL cholesterol in the human, there are differences in the dynamics of their actions. All types of phytosterols lower blood LDL cholesterol by blocking absorption of cholesterol from the intestine.

One difference is that phytosterols tend to have better efficacy at low dose levels than phytostanols (Table 1). This difference was noted by the FDA during issue of the Interim Final Rule for a Health Claim for Sterol/Stanol Esters and Coronary Heart disease (21 CFR Part 101; Sep. 8, 2000). The qualifying dose for sterol esters was 1.3 g sterols esters/day (0.8 g sterols) and 3.4 g of stanol esters (2 g stanols). Mixtures of phytosterols/stanols appear to be intermediate in activity (Table 1).

TABLE 1
Comparison of sterol esters with stanol esters and sterol/stanol mixtures
on low dose efficacy in the reduction of LDL cholesterol.

| | | | | | | Change |
|---|---|---|---|---|---|---|
| | Sterol | Food | Dose | Duration | Change in | in |
| Reference | Type | Matrix | g/day | (days) | Total C | LDL |
| Hendriks. | Sterol | Spread | 0.8 | 25 | −4.3 | −6.2 |
| 1999. | esters | | 1.6 | | −6.2 | −9.2 |
| | | | 3.2 | | −6.8 | −9.8 |
| Beer.2000 | Sterols/ | Milk | 0.9 | 28 | −4.3 | −7.4 |
| | Stanols | drink | 1.8 | | −5.5 | −8.6 |
| | mixture | | 3.6 | | −9.1 | −13.2 |
| Hallikainen. | Stanol | Spread | 0.8 | 28 | NS | NS |
| 2000a | esters | | 1.6 | | −6.8 | −5.6 |
| | | | 2.4 | | −10.3 | −9.7 |
| | | | 3.2 | | −11.3 | −10.4 |

NS = Not statistically significant

Phytostanols are reported to maintain better activity than phytosterols over long term exposures because sterols down regulate bile acid synthesis but not stanols (O'Neil 2005).

Another difference is the effect on endogenous levels of phytosterols in the blood. The body does not synthesize phytosterols. Endogenous phytosterols in the blood are absorbed from various plant materials in the diet. It is known that phytostanols are poorly absorbed whereas sterols, particularly, campesterol, are absorbed to a greater extent. Ingestion of sterols results in an elevation of the blood levels of sitosterol and campesterol. Ingestion of phytostanols has the opposite effect, the levels of sitosterol and campesterol in the blood are depressed. Phytostanols besides blocking the absorption of cholesterol, have a similar effect of blocking the absorption phytosterols.

Mixtures of phytosterols and phytostanols have an intermediate effect with little or no effect on the blood levels of sitosterol and campesterol.

TABLE 2
Comparison Of The Effects Of Sterols, Stanols And Sterol/
Stanol Mixtures On The Blood Levels Of Phytosterols.

| | | | | |
|---|---|---|---|---|
| | | | % Change in | % Change in |
| | | Dose | plasma B- | plasma |
| Publication | Sterol type | g/day | Sitosterol | Campesterol |

STEROLS

| | | | | |
|---|---|---|---|---|
| Westrate. 1998 | Sterol Esters | 3.0 | +39 | +73 |
| Vanstone. 2002 | Sterols | 1.8 | +12 | +72 |
| Ketomaki. 2003 | Sterol Esters | 2.0 | +43 | +53 |
| Amundsen. 2004 | Sterol Esters | 1.2 | +33 | +76 |

STEROL/STANOL MIXTURES

| | | | | |
|---|---|---|---|---|
| Jones. 1999 | Sterol/Stanol | 1.8 | −28 | +4 |
| | mixture | | | |
| Vanstone. 2002 | Sterol/Stanol | 1.8 | +3 | +28 |
| | mixture | | | |

STANOLS

| | | | | |
|---|---|---|---|---|
| Gylling. 1995a | Stanol Esters | 3.0 | −29 | −42 |
| Westrate. 1998 | Stanol Esters | 3.0 | −36 | −17 |
| Vanstone. 2002 | Stanols | 1.8 | −48 | −51 |
| Ketomaki. 2003 | Stanol Esters | 2.0 | −32 | −41 |

Changing blood levels of endogenous phytosterols in the blood may have negative effects on health. Blood levels and intakes of phytosterols are higher in individuals eating a vegetarian diet. This type of diet is associated with a lower risk of heart disease. Low doses of tall oil sterols (sitosterol) has been shown to be effective in reducing the symptoms of benign prostate hypertrophy (Berges.2000; Coleman.2002), a condition that affects the majority of males over the age of 40. There are individual differences in the degree to which phytosterols are absorbed from the diet. There appears to be a positive correlation between blood levels of phytosterols and the development of atherosclerosis (Glueck.1991) and coronary events (Assmann.2006). Although it unlikely that that elevated phytosterols contribute to these correlations (Sudhop.2002), it would nonetheless appear to be an advantage to not significantly raise blood levels of phytosterols. Mixtures of tall oil sterols and tall oil stanols are neutral in their effects on blood levels of sitosterol and campesterol with effects intermediate to that of stanols or sterols alone. The relative amounts of sterols or stanols required depends on the source of the sterols. The optimal percentage of phytostanols in the total mixture of phytosterols is in the range of 14 to 20%. Tall oil sterols have an endogenous content of stanols of about 9 to 12% whereas stanols are usually undetectable in vegetable oil sterols.

To date, most spreads/margarines on the market which are low fat are prepared with phytosterols in the form of esters. However, as described above, there are apparent safety and efficacy advantages to combining both sterol esters and stanol esters.

In addition, the use of 100% phytosterol or phytostanol esters has two disadvantages. One is that the ester forms have poor stability compared to free sterols. The free plant sterols are stable for many years. The ester forms have a shelf life of about 1 year at room temperature when stored under refrigeration and conditions that exclude oxygen.

The second disadvantage is the high melting point of the esters. As esters only become liquid or flowable above 35 to 40 degrees, they require the use of special heating equipment, not normally used in margarine or other food manufacturing to bring the esters to a liquid state. Most food processors lack custom heating equipment to warm up drums of sterol esters. Esters are usually sold in 180 kg or larger size containers. The time required to bring such a container of esters to 40 or 50 degrees from refrigerator temperature is 3 to 6 days which is a highly distinct manufacturing problem.

In view of the difficulties and challenges noted above, and bearing in mind the utility of being able to widely supplement a wide variety of comestible products with healthful plant derived sterols and stanols, it would be highly advantageous to find a way to capitalize on the handling, manufacturing and therapeutic advantages and minimize the disadvantages of both the esterified and non-esterified phytosterol/stanol moieties.

It is an object of the present invention to obviate or mitigate the above noted disadvantages.

### SUMMARY OF THE INVENTION

The present invention provides a composition comprising one or more free (unesterified) phytosterols and/or phytostanols which are substantially completely dissolved in one or more esterified phytosterols and/or phytostanols.

The present invention provides a composition comprising one or more esterified phytosterols and/or phytostanols which are substantially completely dissolved in an edible oil.

The present invention further provides a method of stabilizing from oxidation a composition of one or more esterified phytosterols and phytostanols which comprises solubilizing therein one or more free (unesterified) phytosterols or phytostanols.

The invention further provides a method of maintaining a composition of one or more esterified phytosterols and phytostanols liquid and readily pourable at room temperature which comprises solubilizing therein one or more free (unesterified) phytosterols or phytostanols.

The invention further provides a method of maintaining a composition of one or more esterified phytosterols and phytostanols liquid and readily pourable at room temperature which comprises solubilizing therein a measurable amount of an edible oil.

Surprisingly, it has been found that free, unesterifed phytosterols and phytostanols (preferably phytostanols) can readily be dissolved in phytosterol and/or phytostanol esters by heating the mixture to approximately 90° C. The free phytosterols/stanols remain dissolved once the mixture is cooled. This dissolution in the esters presents a number of advantages. One is that the presence of the free moiety (stanols and sterols) stabilizes from oxidation and rancidity the ester moiety and as such the compositions remain useful for longer post-manufacturing and the foods, beverages and nutraceuticals into which the compositions are incorporated likewise remain useful longer. The other key advantage is that this invention simplifies the process of food, beverage or nutraceutical manufacture. In a preferred form, if an edible oil (for example, a vegetable oil) is added to the composition, the solubility of the free stanols/sterols increases, allowing higher ratios of stanols to sterols in the composition. The edible oil also reduces the melting point of the esters, so that the composition becomes fluid at room temperature, eliminating the need for special warming equipment thereby saving significantly on food, beverage or nutraceutical manufacturing costs. This is a critical advantage which not only assists in manufacturing but reduces materials loss, due to room temperature flowability.

It has been found when as little as 0.3% edible oil (carrying the free sterol/stanol moiety) is added to sterol/stanol esters, and heated as described above, this reduces the melting point of the esters such as the entire composition is flowable at room temperature. The composition is ideally suited for making food products which require the oil solubility of esters such as margarines, salad dressings, and processed cheeses. This is equally applicable to a wide variety of food, beverage, functional food and nutraceutical products. Oils comprising omega-fatty acids can also be used preferentially as the “edible oil” allowing the combination of the heart health benefits of sterols and stanols with that of omega-3-fatty acids.

A further ancillary benefit of the composition of the present invention is the maintenance of a favourable serum phytosterol profile.

The compositions of the present invention have an enormous number of therapeutic uses when administered to animals, in particular humans, not only in respect to the treatment of cardiovascular disease and its underlying conditions such as hypercholesterolemia, hyperlipidemia, atherosclerosis, hypertension, thrombosis but in the treatment and inhibition of other diseases such as Type II diabetes, dementia (including Alzheimer's disease), neural degeneration, cancer (including colon and prostate), and mental disorders such as bipolar disease. In addition, the compositions may be used to enhance brain development and visual acuity.

These effects and other significant advantages will become apparent herein below.

### PREFERRED EMBODIMENTS OF THE INVENTION

The following detailed description is provided to aid those skilled in the art in practising the present invention. However, this detailed description should not be construed so as to unduly limit the scope of the present invention. Modifications and variations to the embodiments discussed herein may be made by those with ordinary skill in the art without departing from the spirit or scope of the present invention. Unless otherwise defined, all technical and scientific terms used herein have the same meaning as commonly understood by one of ordinary skill in the art to which this invention pertains.

As used herein, “animal” means any member of the animal kingdom, including preferably humans.

As used herein, “food” or “food product” or “food material” means any safe, ingestible product for animal use, including human use, and includes “functional foods”, dietary supplements, nutraceuticals, natural health products and “designer foods”.

As used herein, “functional food” means a product that is similar in appearance to conventional foods that is consumed as part of a usual diet, but which has demonstrated physiological benefits and/or reduces the risk of disease.

As used herein, “designer food” has the same meaning as functional food

As used herein, “nutraceutical” means a non-pharmaceutical product prepared in the form of pills, powders, potions and in other medicinal forms not generally associated with food but which has a physiological benefit or provides protection against disease.

Anywhere in the world, nutraceuticals, functional foods, natural health products, and designer foods may be food or food ingredients considered to provide medical or health benefits, including the prevention and treatment of disease.

As used herein, the term “phytosterol” includes all sterols without limitation, for example: sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, and all natural or synthesized forms and derivatives thereof, including isomers. The term “phytostanol” refers to saturated or hydrogenated sterols including all natural or synthesized forms and derivatives thereof, and isomers. It is to be understood that modifications to the phytosterols and phtostanols i.e. to include side chains also falls within the purview of this invention. For example, the purview of this invention clearly includes 24 beta-ethylsitostanol, 24-alpha-ethyl-22-dehydrositostanol. It is also to be understood that, when in doubt throughout the specification, and unless otherwise specified, the term “phytosterol” encompasses both sterol and stanol. In a most preferred form, the sterol is in its saturated form and is a sitostanol, preferably beta-sitostanol.

These sterols and stanols for use in accordance with this invention may be procured from a variety of natural sources. For example, they may be obtained from the processing of plant oils (including aquatic plants) such as corn oil and other vegetable oils, wheat germ oil, soy extract, rice extract, rice bran, rapeseed oil, sunflower oil, sesame oil and fish (and other marine-source) oils. They may also be derived from fungi, for example ergosterol. Accordingly, the present invention is not to be limited to any one source of sterols. U.S. Pat. No. 4,420,427 teaches the preparation of sterols from vegetable oil sludge using solvents such as methanol. Alternatively, phytosterols and phytostanols may be obtained from tall oil pitch or soap, by-products of forestry practises as described, for example, in U.S. Pat. No. 5,770,749, incorporated herein by reference.

Phytosterols and phytostanols, as used herein, may be in their free form or in one or more of their esterified forms i.e. optionally, the phytosterol or phytostanol may be esterified prior to formation of the food products. This esterification step renders the phytosterols and/or phytostanols more soluble in fats and oils which may, in some instances, facilitate the incorporation of the phytosterols into various food products.

To form phytosterol and/or phytostanol esters, many methods are known in the art. For example, one or more suitable aliphatic acids or their esters with low boiling alcohols may be condensed with the selected phytosterol and/or phytostanol. A wide variety of aliphatic acids or their esters may be used successfully and include all aliphatic acids consisting of one or more alkyl chains with one or more terminal carboxyl groups. These aliphatic acids may be natural of synthetic and are represented by the following chemical formulae:

a) R1-COOH (monocarboxylic acid) wherein:
R1 is an unbranched saturated alkyl group, represented by CH₃—, CH₃CH₂— or CH₃(CH₂)ₙCH₂— WHERE n=3-25; or
R1 is a branched saturated alkyl group represented by CₙH₂ₙ₊₁— where n=1-25 is the number of carbon atoms contained in the group R1; the branching typically refers, but is not limited to one or more methyl group side chains (branches); or
R1 is an unbranched or branched unsaturated alkyl group, represented by the formula CₙH₂ₙ₋₂ₘ₊₁, where n=1-25 is the number of carbon atoms in R1 and m=degree of unsaturation; or
   b) HOOC—R2-COOH is a dicarboxylic acid wherein:
R2 is an unbranched saturated alkyl group, represented by —CH₂—, or —CH₂CH₂—, or —CH₂(CH₂)ₙCH₂ where n=3-25; or
R2 is a branched saturated alkyl group represented by —CₙH₂ₙ— where n=1-25 is the number of carbon atoms contained in the group R2; the branching typically refers, but is not limited to, one or more methyl group side chains (branches); or
R2 is an unbranched or branched unsaturated alkyl group, represented by the formula CₙH₂ₙ₋₂ₘ, where n=1-25 is the number of carbon atoms in R2 and m=degree of unsaturation; or
   c) a tricarboxylic acid represented by the formula:

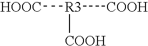

wherein, in this formula:
R3 is a branched saturated alkyl group represented by —CₙH₂ₙ₋₁— where n=1-25 is the number of carbon atoms contained in the group R3; the branching typically refers, but is not limited to, one or more methyl group side chains (branches); or
R3 is a branched unsaturated alkyl group, represented by CₙH₂ₙ₋₂ₘ₋₁— wherein n=1-25 is the number of carbon atoms in R3 and m=the degree of unsaturation; or
   d) a mono-. di-, or tricarboxylic acid as defined above, which may contain one, two or three hydroxyl groups in the molecule.

In a preferred form, the acid is either a straight-chain or branched unsaturated or saturated, aliphatic or aromatic acid. More preferably, the acids are selected, inter alia, from the following list:

valeric acid, isovaleric acid, sorbic acid, isocaproic acid, lauric acid, myrestic acid, palmitic acid, stearic acid, caproic acid, ascorbic acid, arachidic acid, behenic acid, hexacosanoic acid, octacosanoic acid, pentadecanoic acid, erucic acid, linoleic acid, linolenic acid, arachidonic acid, acetic acid, citric acid, tartaric acid, palmitoleic acid and oleic acid. The most preferable fatty acids within the scope of the present invention are linoleic acid, linolenic acid and arachidonic acid which may be obtained from natural sources such as safflower oil, sunflower oil, olive oil and corn oil (linoleic acid), safflower oil, sunflower oil, olive oil and jojoba oil (linolenic acid and arachidonic acid) and rapeseed oil (erucic acid). It is fully contemplated within the scope of the present invention the sterol and stanol esters may be formed with fatty acids selected from: eicosapentaenoic acid (EPA), docosahexanoic acid (DHA), and alpha-linolenic acid (ALA).

Other aromatic acids are clearly contemplated within the scope of the present invention.

By way of example, to form a phytosterol ester, the selected phytosterol and acid or its ester with volatile alcohol may be mixed together under reaction conditions to permit condensation of the phytosterol with the acid. A most preferred method of preparing these esters which is widely used in the edible fat and oil industry is described in U.S. Pat. No. 5,502,045 (which is incorporated herein by reference). As no substances other than the free phytosterol, a fatty acid ester or mixture thereof and an interesterification catalyst like sodium ethylate are used, the technique is highly suitable for preparing products ultimately for human consumption. In overview, this preferred method, adapted for use within the present invention, comprises heating the phytosterol(s) with a vegetable oil fatty acid ester (preferably a methyl ester) at a temperature from 90-120° C. and subsequently adding a suitable catalyst such as sodium ethylate. The catalyst is then removed/destroyed by any one of the techniques known in the art e.g. adding water and/or filtration/centrifugation.

Another method which may be used in accordance with the present invention is described in U.S. Pat. No. 4,588,717, which is also incorporated herein by reference. A preferred method is to mix the phytosterol and the fatty acid together bringing the mixture to a temperature of from about 15° C. to about 45° C. at about atmospheric pressure for approximately one to three hours.

Accordingly, it is to be understood that the widest possible definition is to be accorded to the terms “phytosterol” ester and “phytostanol” ester as used herein, including, but not limited to: esterified phytosterols and phytostanols with aliphatic or aromatic acids (thereby forming aliphatic or aromatic esters, respectively), phenolic acid esters, cinnamate esters, and ferulate esters. It is also to be understood that the term “phytosterols” as used herein, whether singular or plural, unless otherwise indicated, includes both phytosterols and phytostanols.

In a preferred form of the invention, the following ratio of ester:free sterols and/or stanols are contemplated:

| Sterol and/or Stanol Ester (wt %) | Free Sterol and/or Stanol (wt %) |
|---|---|
| 55–99.8 | 0.2–45 |
| 80–99 | 1–20 |
| 90–98 | 2–10 |
| 95–97 | 3–5 |

In a further preferred from, the composition of the present invention comprises from 1-15% by weight unesterified phytosterol and/or phytostanol substantially completely dissolved in 85-99% by weight of one or more esterified phytosterols and/or phytostanols. In a more preferred form the composition comprises from 1-10% by weight unesterified phytosterol and/or phytostanol substantially completely dissolved in 90-99% by weight of one or more esterified phytosterols and/or phytostanols. In a further preferred form, the composition comprises from 4-8% by weight unesterified phytosterol and/or phytostanol substantially completely dissolved in 92-96% by weight of one or more esterified phytosterols and/or phytostanols.

In a preferred form, the composition of the present invention comprises one or more free, unesterifed phytostanols solubilised in phytosterol/phytostanol esters. The key advantage of selecting phytostanols is their advantageous role in depressing the serum levels of sitosterol and campesterol.

In a preferred form of the present invention the composition additionally comprises an edible oil to which the free (unesterifed) phytosterols and/or phytostanols are added prior to the substantially complete dissolution in the esterified phytosterols and/or phytostanols. The edible oil may be selected from but is not limited to the group consisting of olive, rapeseed, canola, sunflower, safflower, sesame, soyabean, corn, coconut, peanut, cottonseed, hemp, flaxseed, and pumpkinseed. Most preferably, the edible oil selected is high in one or more of omega 3 polyunsaturated fatty acids, omega 6 polyunsaturated fatty acids and omega 9 polyunsaturated fatty acids. Such an addition of edible oil reduces the melting point of the esters, so that the composition becomes and stays fluid at room temperature, thereby completely eliminating the need for special warming equipment during processing of the esters into foods and other eivery vehicles.

The compositions of the present invention may be used in a wide variety of foods, beverages and nutraceuticals. The free, unesterified phytosterols/phytostanols may be dissolved into the esters prior to processing into the desired food, beverage or nutraceutical and in fact may be dissolved into the ester during and shortly after the actual ester manufacturing process. In this way, a liquid “premix” composition, comprising free, unesterifed phytosterols and phytostanols (preferably phytostanols), substantially completely dissolved in one or more phytosterol/phytostanol esters would be available for ready and easy transport and use in manufacturing, by remaining liquid at room temperature. In the alternative, the unesterified phytosterols/phytostanols may be dissolved into the esters concomitantly during the production of the particular food, beverage or nutraceutical, as the situation requires.

As well as the wide reaching food and beverage applications, it is to be appreciated that the compositions of the present invention can be readily and beneficially incorporated into nutracuetical deliverables such as gelatine capsules.

### Food Product

Food products include, but are not limited to: milk products, beer, fruit juices, dairy products, margarine and spreads (dairy and non-dairy), peanut and other butters, shortening, sauces, baked goods, fried snack products, confections and chocolate.

Accordingly, examples of food products into which the compositions of the present invention may be incorporated include:

1) Dairy Products—such as cheeses, butter, milk and other dairy beverages, spreads and dairy mixes, ice cream and yoghurt;

2) Fat-Based Products—such as margarines, yellow spreads, mayonnaise, shortenings, cooking and frying oils and dressings;

3) Cereal-Based Products—comprising grains (for example, bread and pastas) whether these goods are cooked, baked or otherwise processed;

4) Confectioneries—such as chocolate, candies, chewing gum, desserts, non-dairy toppings (for example Cool Whip™), sorbets, icings and other fillings;

5) Beverages—dietary supplements and meal replacement drinks such as those sold under the trade-marks BooSt™ and Ensure™; and

6) Miscellaneous Products—including eggs and egg products, processed foods such as soups, pre-prepared pasta sauces, pre-formed meals, potato chips, crackers and snack foods and the like.

While the amount of the composition to be added to the food product will depend on a number of factors, including the type of food product, it is generally preferred that it be in an sufficient amount so that the phytosterol and/or phytostanol (in free and ester form) totals from between 0.05% to 10% by weight of the food product.

### EXAMPLES

The present invention is described by the following non-limiting examples:

### Example 1

### Formulation and Processing for Fat Spreads Containing Stanol—Sterol Ester Mixture

Plain Fat Spread

| | |
|---|---|
| % Sterols as stanols | 20.00 |
| Total sterols and stanols (%) | 7.38 |
| Serving size (g) | 10 |
| Servings per day | 3 |
| Sterol intake g/day | 2.22 |

### Formulation

Fat Phase

| | |
|---|---|
| Canola Oil (Clear Valley 65, Cargill) | 28.227% |
| Sterol Ester (Phytosource) | 11.11% |
| Solid Fat (Magfat CAF 50, Premium Vegetable Oils) | 2.000% |
| Flavour (BuFlaCon 15X fat, DairyChem) | 0.020% |
| Mono- and Di-glycerides (MONO-DI HV 60, Danisco) | 0.300% |
| Lecithin (Leciprime 1800 IPM, Cargill) | 0.080% |
| Stanol (Phytosource) | 0.74% |
| Beta Carotene (22% HS HP, BASF) | 0.002% |

Water Phase

| | |
|---|---|
| Water | 52.081% |
| Buttermilk Powder | 1.000% |
| Pectin (Grindsted Pectin RS 400, Danisco)I | 0.500% |
| Starch (EmTex 12688, Cerestar) | 2.400% |
| Flavour DairyChem (BuFlaCon #200 NND) | 0.040% |
| Citric acid (to pH 4.8) (ADM) | 0.006% |
| Salt (Morton) | 1.500% |

### Procedure

1. Weigh buttermilk powder, starch, citric acid and salt, to create a homogenous powder pre-mix
2. Heat the water to 85° C. and slowly add pectin, stirring with a high shear mixer until solution is clear. Allow to hydrate for 5 minutes
3. Slowly, add powder pre-mix to the pectin solution, and stir well until uniform, using a high shear mixer (make sure there are no clumps left in suspension)
4. Add the water soluble flavour
5. Pasteurize at 60° C. for 15 minutes (or a different combination of time and temperature)
6. Weigh canola oil and add it in a swept surface emulsion tank. Heat the oil to 85° C.
7. Add solid fat and keep mixing until it is completely melted
8. Add stanol an keep mixing until it is completely dissolved
9. Add sterol esters, while mixing continuously
10. After the oil phase becomes clear, add mono & di-glycerides, lecithin, fat soluble flavour and colour, while mixing until homogenous
11. Start adding the water phase into the fat phase, while maintaining the temperature at 65-70° C. and mixing continuously until the emulsion is uniform
12. Pump the emulsified mixture into the swept surface heat exchanger and pinworker
13. Package

### Example 2

Fat Spread Containing Omega-3-Fatty Acids

| | |
|---|---|
| % Sterols as stanols | 20.00 |
| Total sterols and stanols (%) | 7.38 |
| Serving size (g) | 10 |
| Servings per day | 3 |
| Sterol intake g/day | 2.22 |
| % Omega fatty acids | 1.68 |
| Omegas intake g/day | 0.66 |

Formulation

Fat Phase

| | |
|---|---|
| Canola Oil (Clear Valley 65, Cargill) | 25.427% |
| Flaxseed oil (>60% omega-3-fatty acids) (Omegaflo, Omega | 2.800% |
| Nutrition) | |
| Sterol Ester (Phytosource) | 11.11% |
| Solid Fat (Magfat CAF 50, Premium Vegetable Oils) | 2.000% |
| Flavour (BuFlaCon 15X fat, DairyChem) | 0.020% |
| Mono- and Di-glycerides (MONO-DI HV 60, Danisco) | 0.300% |
| Lecithin (Leciprime 1800 IPM, Cargill) | 0.080% |
| Stanol (Phytosource) | 0.74% |
| Beta Carotene (22% HS HP, BASF) | 0.002% |

Water Phase

| | |
|---|---|
| Water | 52.081% |
| Buttermilk Powder | 1.000% |
| Pectin (Grindsted Pectin RS 400, Danisco)l | 0.500% |
| Starch (EmTex 12688, Cerestar) | 2.400% |
| Flavour DairyChem (BuFlaCon #200 NND) | 0.040% |
| Citric acid (to pH 4.8) (ADM) | 0.006% |
| Salt (Morton) | 1.500% |

### Procedure

1. Weigh buttermilk powder, starch, citric acid and salt, to create a homogenous powder pre-mix
2. Heat the water to 85° C. and slowly add pectin, stirring with a high shear mixer until solution is clear. Allow to hydrate for 5 minutes
3. Slowly, add powder pre-mix to the pectin solution, and stir well until uniform, using a high shear mixer (make sure there are no clumps left in suspension)
4. Add the water soluble flavour
5. Pasteurize at 60° C. for 15 minutes (or a different combination of time and temperature)
6. Weigh canola and fish oil and add them in a swept surface emulsion tank. Heat the oil to 85° C.
7. Add solid fat and keep mixing until it is completely melted
8. Add stanol an keep mixing until it is completely dissolved
9. Add sterol esters, while mixing continuously
10. After the oil phase becomes clear, add mono & di-glycerides, lecithin, fat soluble flavour and colour, while mixing until homogenous
11. Start adding the water phase into the fat phase, while maintaining the temperature at 65-70° C. and mixing continuously until the emulsion is uniform
12. Pump the emulsified mixture into the swept surface heat exchanger and pinworker
13. Package

### Example 3

Plain VIVOLA ™ Oil (an oil useful for cooking and dressings etc . . . )

| | |
|---|---|
| % Sterols as stanols | 20.00 |
| Total sterols and stanols (%) | 5.12 |
| Serving size (g) | 12 |
| Servings per day | 3 |
| Sterol intake g/day | 1.84 |

Ingredients

| | |
|---|---|
| MCT Oil (NEOBEE 1053, Stepan) | 63.361% |
| Canola Oil (Safeway) | 13.270% |
| Olive Oil (Bertolli Extra Light) | 11.880% |
| Coconut Oil (Omegaflo, Omega Nutrition) | 6.050% |
| Tall Oil Sterol Esters (Phytosource) | 5.10% |
| Tall Oil Stanols (Phytosource) | 0.34% |

### Procedure

1. Weigh MCT oil and stanol and blend together in a stainless steel vessel. Stir well using a Caframo mixer.
2. Heat the MCT oil with stanol at 70° C. with continuous stirring for 10 minutes until all stanol powder is fully dissolved.
3. Add sterol esters and continue mixing until they are fully dissolved
4. Cool down to 50° C. leaving the mix at room temperature and then add coconut oil, olive oil, canola oil and flaxseed oil in this particular order.
5. Leave the oil blend to cool down at room temperature.
6. Fill the oil into bottles and then seal with caps.
7. Store VIVOLA™ oil at room temperature.

### Example 4

VIVOLA ™ Oil Containing Omega-3-Fatty Acids

| | |
|---|---|
| % Sterols as stanols | 20.00 |
| Total sterols and stanols (%) | 5.12 |
| Serving size (g) | 12 |
| Servings per day | 3 |
| Sterol intake g/day | 1.84 |
| % Omega fatty acids | 3.86 |
| Omegas fatty acids intake g/day | 1.39 |

Ingredients

| | |
|---|---|
| MCT Oil (NEOBEE 1053, Stepan) | 63.361% |
| Olive Oil (Bertolli Extra Light) | 11.880% |
| Canola Oil (Safeway) | 6.830% |
| Flaxseed Oil (Omegaflo, Omega Nutrition) | 6.440% |
| Coconut Oil (Omegaflo, Omega Nutrition) | 6.050% |
| Tall Oil Sterol Esters (Phytosource) | 5.10% |
| Tall Oil Stanols (Phytosource) | 0.34% |

### Procedure

1. Weigh MCT oil and stanol and blend together in a stainless steel vessel. Stir well using a Caframo mixer.
2. Heat the MCT oil with stanol at 70° C. with continuous stirring for 10 minutes until all stanol powder is fully dissolved.
3. Add sterol esters and continue mixing until they are fully dissolved
4. Cool down to 50° C. leaving the mix at room temperature and then add coconut oil, olive oil, canola oil and flaxseed oil in this particular order.
5. Leave the oil blend to cool down at room temperature.
6. Fill the oil into bottles and then seal with caps.
7. Store VIVOLA™ oil at room temperature.
8. Attention! This oil may show some precipitation if it will be stored at temperatures below 8° C.

### Example 5

Chewable Candies

| | |
|---|---|
| % Sterols as stanols | 20.00 |
| Total sterols and stanols (%) | 5.12 |
| Serving size (g) | 12 |
| Servings per day | 3 |
| Sterol intake g/day | 1.84 |

Ingredients

| | |
|---|---|
| Corn syrup (White, Crown Lily) | 38.880% |
| Sugar (Golden Yellow, Rogers) | 34.990% |
| Water | 10.144% |
| Tall Oil Sterol Esters (Phytosource) | 7.69% |
| Corn starch (Safeway) | 4.670% |
| Skim milk powder (Safeway) | 3.110% |
| Tall Oil Stanols | 0.51% |

### Procedure

1. Weigh corn syrup and sugar into a saucepan. Warm up and mix until all sugar is melted.
2. Add the sterol esters into the syrup mixture, and keep mixing until they are fully dissolved.
3. Weigh milk powder, corn starch and stanol, and add them into the water. Blend them homogenously.
4. Heat the water mixture to 70-80 C.
5. Transfer the water mixture into the syrup mixture.
6. Heat the mixture and keep stirring up until the temperature reaches 121 C.
7. Pour the mixture into a Pyrex baking tray. Cool it down to ˜35 C.
8. Cut into squares and wrap in waxed paper.

### Example 6

Processed Cheddar Cheese

| | |
|---|---|
| % Sterols as stanols | 20.00 |
| Total sterols and stanols (%) | 5.98 |
| Serving size (g) | 30 |
| Servings per day | 1 |
| Sterol intake g/day | 1.80 |

Ingredients

| | |
|---|---|
| Cheddar Cheese Curd (Laack Cheese, WI) | 88.15% |
| Tall Oil Sterol Esters (Phytosource) | 9.00% |
| Disodium Phosphate (DSP, Rhodia Foods) | 1.5% |
| Trisodium Phosphate (TSP, Rhodia Foods) | 0.75% |
| Tall Oil Stanols (Phytosource) | 0.60% |

### Procedure

1. Add Cheddar curd, stanol powder, sterol esters and stabilizers (i.e. DSP and TSP) to the cheese cooker (all at once) and start heating directly to ˜76° C. (169° F.), while mixing continuously. Equipment: Custom Stainless Equipment Co. Santa Rosa, Calif. 95401, Model No: CDB-0718 FVX, Serial No: 82079
2. Mold/Package in 5 lb loaf boxes with liners
3. Store in cooler

### Example 7

Chocolate

| | |
|---|---|
| % Sterols as stanols | 20.00 |
| Total sterols and stanols (%) | 5.21 |
| Serving size (g) | 12 |
| Servings per day | 3 |
| Sterol intake g/day | 1.87 |

Ingredients

| | |
|---|---|
| Regular/Sugar Free Dark Chocolate (Schokinag) | 86.440% |
| Tall Oil Sterol Esters (Phytosource) | 7.83% |
| Liquid Coconut Oil (Kokoheart-23, Bunge | 3.095% |
| Foods/Solae) | |
| Butter Oil (Lactantia, Parmalat) | 1.895% |
| Tall Oil Stanols (Phytosource) | 0.52% |
| Lecithin (Canasperse U.B. F. -K, Bunge Foods/ | 0.220% |
| Solae) | |

### Procedure

1. Filling Mixture:
a. Weigh 25,000 g chocolate and mix it at 28 C with stanols, coconut oil, lecithin, and half of sterol esters. Continue mixing until homogenous.

2. Chocolate Shells:
a. Melt the rest of the chocolate in designated melter, then add the butter oil and the rest of sterol esters. Mix well until uniform.
b. Pump melted chocolate to the designated tempering unit.
c. Pump tempered chocolate to the depositing stations of the moulding machines

3. Add the filling mixture into the temperate chocolate moulds

4. Cool down the mould through a cooling tunnel

5. Package the chocolate

### Example 8

Mayonnaise

| | |
|---|---|
| % Sterols as stanols | 20.00 |
| Total sterols and stanols (%) | 3.99 |
| Serving size (g) | 15 |
| Servings per day | 3 |
| Sterol intake g/day | 1.79 |

Ingredients

| | |
|---|---|
| Tall Oil Stanols (Phytosource) | 0.40% |
| Tall Oil Sterol Esters (Phytosource) | 6.00% |
| Canola oil (Safeway) | 43.721% |
| Fresh Egg Yolk (Safeway) | 21.930% |
| Water | 15.020% |
| Modified Starch (‘Textra’ - NACAN) | 3.880% |
| Vinegar (Safeway) | 3.680% |
| Mustard (Safeway) | 2.520% |
| Lemon Juice (Safeway) | 2.100% |
| Salt (Morton) | 0.750% |

### Procedure

1. Weigh canola oil and stanol and blend together in a stainless steel vessel. Stir well using a Caframo mixer.
2. Heat the canola oil with stanol at 70° C. with continuous stirring for 10 minutes until all stanol powder is fully dissolved.
3. Add sterol esters and continue mixing until they are fully dissolved
4. Mix starch with cold water to create a thick slurry (20.54%). Heat the slurry at high temperature until it starts boiling and becomes a transparent paste
5. Separately, mix the egg yolks slowly, for 1-2 minutes
6. Slowly, start adding the oil, while also adding the starch paste, little by little. Continue to alternate mixing in the oil and the starch paste until they are both uniformly incorporated into the mayonnaise
7. Add the vinegar, mustard, lemon juice and salt, one by one, and mix well until uniform

### Example 9

Ranch Dressing

| | |
|---|---|
| % Sterols as stanols | 20.00 |
| Total sterols and stanols (%) | 2.00 |
| Serving size (g) | 30 |
| Servings per day | 3 |
| Sterol intake g/day | 1.80 |

Ingredients

| | |
|---|---|
| Butter Milk | 46.93% |
| Canola oil | 20.70% |
| Vinegar | 7.75% |
| Skim milk powder | 7.49% |
| Buttermilk Powder | 3.14% |
| Tall Oil Sterol Ester | 3.01% |
| Fresh Egg Yolk | 2.33% |
| Parmesan | 2.01% |
| Whey protein | 1.66% |
| Lemon juice | 1.24% |
| Salt | 0.99% |
| Butter Buds - High | 0.93% |
| Concentrate | |
| Sugar | 0.59% |
| TIC Gum - Saladizer 250 | 0.20% |
| Onion powder | 0.19% |
| Garlic powder | 0.19% |
| Sodium Benzoate | 0.15% |
| Tall Oil Stanol | 0.20% |
| Parsley | 0.09% |
| Chives | 0.09% |
| Oregano | 0.09% |
| White pepper | 0.04% |

### Procedure

1. Weigh canola oil and stanol and blend together in a stainless steel vessel.
2. Stir well using a Caframo mixer.
3. Heat the canola oil with stanol at 70° C. with continuous stirring for 10 minutes until all stanol powder is fully dissolved.
4. Add sterol esters and continue mixing until they are fully dissolved
5. Mix slowly egg yolk with gum, then start adding canola oil (prepared above) little by little
6. Slowly, start adding the buttermilk, and continue mixing until homogenous Separately, mix all dry ingredients, then slowly start adding the powder mix into the liquid mix above
7. Add vinegar and lemon juice

### Example 10

Granola Bar

| | |
|---|---|
| % Sterols as stanols | 20.00 |
| Total sterols and stanols (%) | 7.18 |
| Serving size (g) | 25 |
| Servings per day | 1 |
| Sterol intake g/day | 1.80 |

| Ingredients | % |
|---|---|
| Cereals | |
| Quick Oats | 30.970 |
| Crisp Rice | 10.700 |
| Coconut | 6.280 |
| Binder | |
| Tall Oil Stanol | 0.72 |
| Tall Oil Sterol Ester | 10.80 |
| Canola Oil | 3.330 |
| White Sugar | 7.500 |
| Molasses | 3.000 |
| Corn Syrup | 25.500 |
| Salt | 0.200 |
| Vanilla Flavour | 0.800 |
| SSL | 0.200 |

### Procedure

1. Mix cereals and coconut
2. Melt stanol in oil bringing to a boil. Take pot from heat source, add sterol esters, honey, molasses and sugar and
3. Mix well, while binder is cooling down and becomes thicker
4. Add flavour to the binder and mix well.
5. Add binder to cereal mixture while hot (60-70 C), otherwise it will be too tough and will not allow mixing
6. Mix well and roll in a mold.
7. Cut the bars, then cool them down and pack

Examples 11—describes the use of liquid “premixes” of the composition of the present invention, in the preparation of various foods and nutraceuticals:

### Example 11

### Foods Formulated With Pre-Mixes

TABLE 1
Examples Of 100 kg of Pre-Mixes For Foods

| | PREMIX | | | |
|---|---|---|---|---|
| | A | B | C | D |
| Free Stanols | 1.77 | 1.39 | 4.22 | 4.22 |
| Sterol Esters | 97.73 | 76.68 | 61.63 | 61.63 |
| Omega fatty acids | 0.00 | 21.93 | 0.00 | 34.15 |
| Canola Oil | 0.50 | 0.00 | 34.15 | 0.00 |
| Total (kg) | 100 | 100 | 100 | 100 |
| Percent of total sterols as | 97.21 | 97.21 | 90.23 | 90.23 |
| esters | | | | |
| Percent of total sterols as | 14.00 | 14.00 | 20.00 | 20.00 |
| stanols | | | | |

All pre-mixes are prepared under conditions that exclude oxygen; usually with a nitrogen blanket. All omega fatty acids have been deodourized prior to use. The omega fatty acids can be either purified omega fatty acid from fish oil (>60% omegas) or flax seed oil (>60% alpha-linoleic acid). The sterol esters have a sterol content of >60%.

### Method of Preparation No. 1:

1. Heat the sterol esters to 90 degrees, with mixing add vegetable oil and or omega fatty acids and stanols.
2. Fill mixtures into food grade steel drums under a nitrogen blanket.
3. Store mixtures at refrigerator temperature.

### Method of Preparation No. 2

1. The esters at the end of the manufacturing process are at temperatures above 100 degrees. The esters are then passed through a deodourizing process.
2. The exit stream is blended with stanols that have been dissolved at 90 degrees in vegetable oil and or omega fatty acids.
3. Fill mixtures into food grade steel drums under a nitrogen blanket.
4. Store mixtures at refrigerator temperature.

Margarines - Dosages, Stanols And Omega Fatty Acids Content

PREMIX

| | A | B | C | D |
|---|---|---|---|---|
| Servings per day | 3 | 3 | 3 | 3 |
| Serving Size (g) | 10.0 | 10.0 | 10.0 | 10.0 |
| % Total Sterols in food | 6.00 | 6.00 | 6.00 | 6.00 |
| Gm Sterols per day | 1.80 | 1.80 | 1.80 | 1.80 |
| Gm Omega-FA per day | 0.00 | 0.50 | 0.00 | 0.90 |

TABLE 3
Formulations of Margarines

| Fat Phase (Total 42.5 kg) | A | B | C | D |
|---|---|---|---|---|
| Kg of pre-mix/100 kg | 9.9 | 12.7 | 14.6 | 14.6 |
| Canola oil | 18.3 | 15.6 | 13.6 | 13.6 |

Solid fat, 2.0 kg; Flavour, 0.02 kg; mono- and diglycerides, 0.30 kg; lecithin, 0.08 kg, beta-carotene 0.002 kg.

Water Phase: (Total 57.5 kg) water, 52.1 kg; Buttermilk powder, 1.0 kg; Pectin, 0.5 kg; Starch, 2.4 kg; Flavour, 0.04 kg; citric acid, 0.006 kg, salt, 1.5 kg

### Method of Preparation (as Described above in Previous Examples with Premix Added at Steps 8/9)

TABLE 4
Mayonaises And Salad Dressings, Dosages, Stanols And Omega Fatty
Acids Content

PREMIX

| | A | B | C | D |
|---|---|---|---|---|
| Servings per day | 3.00 | 3.00 | 3.00 | 3.00 |
| Serving Size (g) | 15.00 | 15.00 | 15.00 | 15.00 |
| % Total Sterols in food | 4.00 | 4.00 | 4.00 | 4.00 |
| % total sterols as stanols | 14.00 | 14.00 | 20.00 | 20.00 |
| Gm Sterols per day | 1.80 | 1.80 | 1.80 | 1.80 |
| Gm Omega-FA per day | 0.00 | 0.50 | 0.00 | 0.50 |
| Kg of pre-mix/100 kg of food | 6.60 | 8.42 | 15.31 | 15.31 |

TABLE 5
Mayonaises And Salad Dressings Formulations

PREMIX

| | A | B | C | D |
|---|---|---|---|---|
| Kg of pre-mix/100 kg of food | 6.60 | 8.42 | 15.31 | 15.31 |

TABLE 6
Processed Cheese, Dosages, Stanols And Omega Fatty Acids

| | A | B | C | D |
|---|---|---|---|---|
| Servings per day | 3.00 | 3.00 | 3.00 | 3.00 |
| Serving Size (g) | 30.00 | 30.00 | 30.00 | 30.00 |
| % Total Sterols in food | 2.00 | 2.00 | 2.00 | 2.00 |
| % total sterols as stanols | 14.00 | 14.00 | 20.00 | 20.00 |
| Gm Sterols per day | 1.80 | 1.80 | 1.80 | 1.80 |
| Gm Omega-FA per day | 0.00 | 0.50 | 0.00 | 0.50 |
| Kg of pre-mix/100 kg of | 3.30 | 4.21 | 7.66 | 7.66 |
| cheese | | | | |

TABLE 7
Processed Cheese formulation

| | A | B | C | D |
|---|---|---|---|---|
| Kg of pre-mix/100 kg of | 3.30 | 4.21 | 7.66 | 7.66 |
| cheese | | | | |

TABLE 8
Chocolates pre-mixes for formulations - 100 kg

| | E |
|---|---|
| Stanols | 3.00 |
| Sterol Esters | 70.89 |
| Coconut oil | 14.59 |
| Butter Oil | 10.83 |
| Lecithin | 0.69 |

TABLE 9
Chocolates, dosages, and stanols using pre-mixes

| | E |
|---|---|
| Servings per day | 3 |
| Serving Size (g) | 10.0 |
| % Total Sterols in food | 6.00 |
| % total sterols as stanols | 17.00 |
| % Total phytosterols as esters | 93.72 |
| Gm Sterols per day | 1.80 |
| Gm Omega-FA per day | 0.00 |
| Kg of pre-mix/100 kg of | 13.2 |
| Chocolate | |

TABLE 10
Chocolate Filling Formulation

| | E |
|---|---|
| Kg of pre-mix/100 kg of Chocolate | 13.2 |

TABLE 11
Examples of softgels with sterols esters and free tall oil stanols,
dosages and stanols content

| | Formulation | | | |
|---|---|---|---|---|
| | E | F | G | H |
| Capsules per day | 4 | 4 | 4 | 6 |
| Gm sterols per day | 1.8 | 1.8 | 1.8 | 1.8 |
| Gm sterols per capsule | 0.450 | 0.450 | 0.450 | 0.300 |
| % Total sterols as esters | 97.21 | 94.88 | 92.56 | 90.23 |
| % Total sterols as | 14.00 | 16.00 | 18.00 | 20.00 |
| stanols | | | | |

TABLE 12
100 kg batches of fill material

Formulation

| | E | F | G | H |
|---|---|---|---|---|
| Free Stanols (kg) | 1.76 | 3.10 | 3.64 | 4.00 |
| Sterol Esters (kg) | 97.24 | 90.90 | 71.56 | 58.48 |
| Vegetable oil (kg) | 1.00 | 6.00 | 24.80 | 37.52 |
| Total Weight (kg) | 100 | 100 | 100 | 100 |

TABLE 13
Soft gelatin capsules fill weights

Formulation

| | E | F | G | H |
|---|---|---|---|---|
| Gm free stanols per | 0.013 | 0.024 | 0.035 | 0.031 |
| capsule | | | | |
| Gm sterol esters per | 0.729 | 0.712 | 0.694 | 0.451 |
| capsule | | | | |
| Gm vegetable oil per | 0.007 | 0.047 | 0.241 | 0.290 |
| capsule | | | | |
| Fill weight of capsule | 0.750 | 0.783 | 0.970 | 0.772 |
| (gm) | | | | |

The fill material with formulation E was somewhat viscous and recoveries of capsules were relatively low. The higher proportions of vegetable oil as shown in formulations F, G, and H will result in a more liquid fill and higher recoveries. The solubility of the stanols was about 3% in the esters, but was over 4% in corn oil used in this example.

Procedure: The stanols component was blended with the vegetable oil in a cobalt mill and then mixed with esters which had been warmed to 40 degrees.

Capsule material contained beef gelatin (BSE free), glycerin, water, titanium oxide masking agent, and light yellow colouring agent.

Although beef gelatin was used in formulation E, other gelatins such as, pork, fish or non-animal (plant) gelatins may also easily be used.

### Example 12 (Dissolution of Stanols in Esters)

Composition of:

0.344% stanol

11.5% sterol esters

### Stanols

Code: FCP-3P2

BRI ID: FM-P2-83

Manuf.lot# 5QC27H-2

### Description (Dry Form, Room Temp):

white powder composed of longitudinally shaped granules/crystals. White flour-like feel.

### Sterol Esters

### Description:

Temp (−) 18 C—solid

Temp 13-15 C—sticky texture, viscosity less than that of, for example, honey at room temperature, therefore has a semi-high viscosity. Able to be pumped?—no

Temp 35 C-viscosity—slightly more viscous than regular canola oil at room temp
appearance—resembles canola oil (clear, golden color)

### Temp 60 C viscosity—that of canola oil at room temp, however more sticky to touch than normal oil

### Stanols with Sterol Esters Description

1. 0.344 g stanol added to 11.5 g sterol esters. Sterol ester increased to temp 90° C.

Stanols dissolved fully
2. Stanol/sterol esters, fully dissolved, cooled to 35 C

No apparent recrystallization of stanols into its original crystals. Sterol esters appear smooth, no appearance of crystals within
3. Stanol/sterol esters, same sample as above, cooled to 25 C

Stanols dissolved fully, Good appearance and handling.

End Composition: 97% esters and 3% stanols

### REFERENCES

1. Law M. R., Wald N. J., Wu., Hacksaw Z A., Bailey A.; Systemic underestimation of association between serum cholesterol concentration and ischemic heart disease in observational studies: Data from BUPA Study; Br. Med. J. 1994; 308:363-366
2. Law M. R., Wald N. J., Thompson S. G.; By how much and how quickly does reduction in serum cholesterol concentration lower risk of ischemic heart disease? Br. Med. J. 1994; 308:367-373
3. Kannel W B, Castelli W P, Gordon T et al. Lipoprotein cholesterol in the prediction of atherosclerotic disease: new perspectives based on the Framingham Heart Study. Ann Intern Med. 1995; 90:85-91
4. Singh B K, Mehta J L. Management of dyslipidemia in the primary prevention of coronary heart disease. Curr Opin Cardiol. 2002; 17:503-11
5. La Rosa J. C., Hunninghake D. Bush D. et al.; The cholesterol facts: A summary of the evidence relating to dietary fats, serum cholesterol and coronary heart disease: A joint statement by the American Heart Association and the National Heart, Lung and Blood Institute. Circulation 1990; 81:1721-1733
6. Havel R. J., Rapaport E. Drug Therapy: Management of Primary Hyperlipidemia. New England Journal of Medicine, 1995; 332:1491-1498
7. Kuccodkar et al.; Effects of plant sterols on cholesterol metabolism. Atherosclerosis, 1976; 23:239-248
8. Lees R. S., Lees A. M. Effects of sitosterol therapy on plasma lipid and lipoprotein concentrations. In: Greten H (Ed) Lipoprotein Metabolism. Springer-Verlag, Berlin, Heidelberg, New York, 1976:119-124
9. Lees A. M., Mok H. Y. I., Lees R. S., McCluskey M. A., Grundy S. M. Plant sterols as cholesterol-lowering agents: clinical trials in patients with hypercholesterolemia and studies of sterol balance. Atherosclerosis 1977; 28: 325-338

### FURTHER REFERENCES

Amundsen A L, Ntanios F, Put N, Ose L.
   Long-term compliance and changes in plasma lipids, plant sterols and carotenoids in children and parents with FH consuming plant sterol ester-enriched spread. Eur J Clin Nutr. 2004 December; 58(12):1612-20.
Assmann G, Cullen P, Erbey J, Ramey D R, Kannenberg F, Schulte H. Plasma sitosterol elevations are associated with an increased incidence of coronary events in men: results of a nested case-control analysis of the Prospective Cardiovascular Munster (PROCAM) study. Nutr Metab Cardiovasc Dis. 2006 January; 16(1):13-21.
Berges R R, Kassen A, Senge T. Treatment of symptomatic benign prostatic hyperplasia with beta-sitosterol: an 18-month follow-up. BJU Int. 2000 May; 85(7):842-6.
Coleman C I, Hebert J H, Reddy P. The effect of phytosterols on quality of life in the treatment of benign prostatic hyperplasia. Pharmacotherapy. 2002 November; 22(11):1426-32
Glueck C J, Speirs J, Tracy T, Streicher P, Illig E, Vandegrift J.
   Relationships of serum plant sterols (phytosterols) and cholesterol in 595 hypercholesterolemic subjects, and familial aggregation of phytosterols, cholesterol, and premature coronary heart disease in hyperphytosterolemic probands and their first-degree relatives. Metabolism. 1991 August; 40(8):842-8.
Gylling H, Siimes M A, Miettinen T A. Sitostanol ester margarine in dietary treatment of children with familial hypercholesterolemia. J Lipid Res. 1995 August; 36(8):1807-12.
Hallikainen M A, Sarkkinen E S, Uusitupa M I. Plant stanol esters affect serum cholesterol concentrations of hypercholesterolemic men and women in a dose-dependent manner. J Nutr. 2000 April; 130(4):767-76.
Hendriks H F, Weststrate J A, van Vliet T, Meijer G W. Spreads enriched with three different levels of vegetable oil sterols and the degree of cholesterol lowering in normocholesterolaemic and mildly hypercholesterolaemic subjects.

### Eur J Clin Nutr. 1999 April; 53(4):319-27.

Jones P J, Ntanios F Y, Raeini-Sarjaz M, Vanstone C A.

### Cholesterol-lowering efficacy of a sitostanol-containing phytosterol mixture with a prudent diet in hyperlipidemic men.

### Am J Clin Nutr. 1999 June; 69(6):1144-50.

Ketomaki A M, Gylling H, Antikainen M, Siimes M A, Miettinen T A.

### Red cell and plasma plant sterols are related during consumption of plant stanol and sterol ester spreads in children with hypercholesterolemia.

### J. Pediatr. 2003 May; 142(5):524-31.

O'Neill F H, Sanders T A, Thompson G R. Comparison of efficacy of plant stanol ester and sterol ester: short-term and longer-term studies. Am J Cardiol. 2005 Jul. 4; 96(1A):29D-36D.
Scientific Committee on Food Report: General view of the Scientific Committee on Food on the long-term effects of the intake of elevated levels of phytosterols from multiple dietary sources, with particular attention to the effects on β-carotene. SCF/CS/NF/20 ADD 1 Final 3 Oct. 2002.
Sudhop T, Gottwald B M, von Bergmann K. Serum plant sterols as a potential risk factor for coronary heart disease. Metabolism. 2002 December; 51 (12):1519-21.
Vanstone C A, Raeini-Sarjaz M, Parsons W E, Jones P J. Unesterified plant sterols and stanols lower LDL-cholesterol concentrations equivalently in hypercholesterolemic persons. Am J Clin Nutr. 2002 December; 76(6):1272-8.
Weststrate J A, Meijer G W. Plant sterol-enriched margarines and reduction of plasma total- and LDL-cholesterol concentrations in normocholesterolaemic and mildly hypercholesterolaemic subjects. Eur J Clin Nutr. 1998 May; 52(5):334-43.

We claim:

1. A composition comprising one or more free (unesterified) phytosterols and/or phytostanols which are substantially completely dissolved in one or more esterified phytosterols and/or phytostanols.

2. The composition of claim 1 comprising free (unesterified) phytostanol which is substantially completely dissolved in one or more esterified phytosterols and/or phytostanols.

3. The composition of claim 1 comprising from 1-15% by weight unesterified phytosterol and/or phytostanol substantially completely dissolved in 85-99% by weight of one or more esterified phytosterols and/or phytostanols.

4. The composition of claim 1 comprising from 1-10% by weight unesterified phytosterol and/or phytostanol substantially completely dissolved in 90-99% by weight of one or more esterified phytosterols and/or phytostanols.

5. The composition of claim 1 comprising from 4-8% by weight unesterified phytosterol and/or phytostanol substantially completely dissolved in 92-96% by weight of one or more esterified phytosterols and/or phytostanols.

6. The composition of claim 1 wherein the composition additionally comprises an edible oil to which the free (unesterifed) phytosterols and/or phytostanols are added prior to the substantially complete dissolution in the esterified phytosterols and/or phytostanols.

7. The composition of claim 6 wherein the edible oil is selected from the group consisting of olive, rapeseed, canola, sunflower, safflower, sesame, soyabean, corn, coconut, peanut, cottonseed, hemp, flaxseed, and pumpkinseed.

8. The composition of claim 1 incorporated into foods, beverages and nutraceuticals.

9. The composition of claim 1 wherein the edible oil is high in one or more of omega 3 polyunsaturated fatty acids, omega 6 polyunsaturated fatty acids and omega 9 polyunsaturated fatty acids.

10. The composition of claim 1 wherein the edible oil is of marine or fish origin.

11. The composition of claim 1 supplemented with omega fatty acids derived from microalgae.

12. The composition of claim 1 wherein the phytosterols and phytostanols are derived from either tall oil or vegetable oil sources.

13. A method of stabilizing from oxidation a composition of one or more esterified phytosterols and phytostanols which comprises solubilizing therein one or more free (unesterified) phytosterols or phytostanols.

14. The method of claim 13 wherein one or more free phytostanols are solubilised therein.

15. The method of claim 13 wherein from 1-15% by weight unesterified phytosterol and/or phytostanol is solubilised in 85-99% by weight of one or more esterified phytosterols and/or phytostanols.

16. A method of maintaining a composition of one or more esterified phytosterols and phytostanols liquid and readily pourable at room temperature which comprises solubilizing therein one or more free (unesterified) phytosterols or phytostanols.

17. The method of claim 16 wherein the free (unesterified) phytosterols or phytostanols are provided for solubilization in a small amount of an edible oil carrier.

18. The composition of claim 1 wherein the phytosterol is selected from the group consisting of sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol and all natural or synthesized forms and derivatives thereof, including isomers.

19. The composition of claim 1 wherein the phytostanol is selected from the group consisting of all saturated or hydrogenated phytosterols and all natural or synthesized forms and derivatives thereof, including isomers.

20. The composition claim 1 wherein the esters of phytosterols and/or phytostanols are in form selected from the group consisting of: aliphatic acid esters, aromatic acid esters, phenolic acid esters, cinnamate esters, ferulate esters, phytosterol/phytostanol glycosides, and phytosterol/phytostanol acylglycosides.

21. The composition of claim 8 wherein the phytosterol and/or phytostanol (in free and ester form) is added to in an amount totalling from between 0.05% to 10% by weight of the food or beverage product.

22. The composition of claim 8 wherein the food product is fat-based.

23. The composition of claim 8 wherein the food is selected from the group consisting of dairy products, margarine, spreads, peanut and other butters, shortening, meats, poultry, seafood, sauces, fruits, vegetables, grains, grain-derived products, baked goods, fried snack products, confections and chocolate.
